(19) United States
(12) Patent Application Publication
(10) Pub. No.: US 2022/0396766 A1
(43) Pub. Date: Dec. 15, 2022

(54) METHOD FOR ARTERIAL ENDOTHELIAL-ENHANCED FUNCTIONAL T CELL GENERATION

(71) Applicant: Institute of Hematology and Blood Diseases Hospital, CAMS & PUMC, Tianjin (CN)

(72) Inventors: Tao CHENG, Tianjin (CN); Jun SHEN, Tianjin (CN); Jianxiang WANG, Tianjin (CN); Zack Z. WANG, Reading, MA (US); Min WANG, Tianjin (CN); Yingxi XU, Tianjin (CN); Shuo ZHANG, Tianjin (CN); Shuzhen LV, Tianjin (CN)

(73) Assignee: Institute of Hematology and Blood Diseases Hospital, CAMS & PUMC, Tianjin (CN)

(21) Appl. No.: 17/513,918

(22) Filed: Oct. 29, 2021

(30) Foreign Application Priority Data
Jun. 11, 2021 (CN) 202110656914.3

(51) Int. Cl. C12N 5/071

(52) U.S. Cl. CPC C12N 5/069; C12N 2501/125; C12N 2501/145; C12N 2501/2303; C12N 2501/165; C12N 2501/115; C12N 2501/155

(57) ABSTRACT

A method for arterial endothelial-enhanced functional T cell generation is provided. In the method, arterial endothelial cells enhance functional T cell generation by promoting the generation of hematopoietic progenitor cells with T-lineage bias. The first stage of T cell differentiation from human pluripotent stem cells (hPSCs) is optimized, and it is found that hPSC-derived autologous arterial endothelial cells increase the T cell potential of hematopoietic progenitor cells. Moreover, the T cells generated by arterial endothelial cell priming share similar function to that of human peripheral blood T cells. hPSC-derived CD19-CAR-T cells have been verified to have tumor-killing effects both in vivo and in vitro. The established hPSC-T differentiation system would provide a valuable resource for chimeric antigen receptor T cell (CAR-T) therapy.

### CROSS-REFERENCE TO RELATED APPLICATIONS

This application claims priority to Chinese Patent Application No. 202110656914.3 with a filing date of Jun. 11, 2021. The content of the aforementioned application, including any intervening amendments thereto, is incorporated herein by reference.

### TECHNICAL FIELD

The present disclosure relates to the field of biomedicine, and in particular to a method for arterial endothelial-enhanced functional T cell generation.

### BACKGROUND ART

T cells are important immune cells for disease infection resistance and anti-tumor in human body and have various biological functions, such as directly killing target cells, assisting or inhibiting the production of antibodies by B cells and the like. Chimeric antigen receptor T cell (CAR-T) immunotherapy is a novel and precise targeted therapy that can accurately, rapidly and efficiently treat or even may cure cancers. In recent years, CD19-CAR-T has achieved desirable effects in the treatment of B-cell acute lymphoblastic leukemia (B-ALL). At present, one of the major shortcomings of CAR-T therapy is insufficiency of T cells. According to statistics, 30%-50% patients are accompanied by T cell exhaustion, and this greatly limits the use of T cells in tumor immunotherapy.

Human pluripotent stem cells (hPSCs), including embryonic stem cells (ESCs) and induced pluripotent stem cells (iPSCs), are multipotential, self-renewing cells with the ability to differentiate into various types of blood cells in vitro, offering powerful resource for the treatment of blood diseases. Since the first study of hematopoietic differentiation of human ESCs in 2001 by Kaufman et al., a broad spectrum of hPSC-derived blood cell lineages such as red blood cells, T cells and megakaryocytes and the like have been conducted. However, in the past 20 years, it is still a big challenge to obtain functional T lymphocytes stably and efficiently in vitro.

The differentiation of hPSCs into T cells mainly includes two steps: inducing the generation of hematopoietic progenitor cells (HPCs) as the first stage, and inducing the differentiation of HPCs into T cells as the second stage.

In the first stage, embryoid body (EB) formation, stromal cell co-culture and monolayer culture are commonly used methods to induce the generation of HPCs from hPSCs. hPSCs can automatically aggregate to form a cell cluster with a three-germ-layer-like structure to further induce the generation of HPCs, namely the EB formation. The HPCs can also be obtained by digesting the hPSCs into an appropriate size and then co-culturing on mouse OP9 cells or other stromal cells, namely the stromal cell co-culture. The monolayer culture is to directly plate the single hPSCs in specific extracellular matrix protein-coated plates for hematopoietic differentiation. Generally, regardless of the above methods, the hematopoietic differentiation at this stage tends to be primitive hematopoiesis with a limited T cell differentiation potential.

At present, the studies on T cell differentiation mainly focus on the second stage where the stromal cell co-culture is commonly used for inducing HPCs to differentiate into T cells. The most commonly-used stromal cells are OP9 cells or MS5 cells overexpressing the Notch ligands such as DLL1 or DLL4. There are two specific co-culture methods for inducing T cell differentiation from HPCs. One is 2D co-culture, where the HPCs are inoculated on stromal cells, and the stromal cells are continuously renewed to induce the T cell differentiation; the other is 3D co-culture, where the hPSCs or the HPCs were centrifuged with the stromal cells to form small 3D aggregates to simulate the thymic microenvironment for inducing the T cell differentiation. Generally, the 3D co-culture method is better than the 2D co-culture method and can produce more T cells having a certain function. However, complete T cell function still needs to be further evaluated.

Existing studies on T cell differentiation mainly focus on the second stage, while ignoring the first stage where the generated HPCs bias towards primitive hematopoiesis to cause the T cells produced in the second stage with relatively small number and incomplete functions.

### SUMMARY

The purpose of the present disclosure is to overcome the shortcomings of the prior art and provides a method for arterial endothelial-enhanced functional T cell generation.

The present disclosure adopts the following technical solutions:

The present disclosure provides use of arterial endothelial cells in enhancing functional T cell generation.

Preferably, the arterial endothelial cells may enhance functional T cell generation by promoting the generation of HPCs with T-lineage bias.

Preferably, the arterial endothelial cells may be autologous arterial endothelial cells.

Preferably, the arterial endothelial cells may be hPSC-derived autologous arterial endothelial cells.

The present disclosure further provides a method for arterial endothelial-enhanced functional T cell generation, including the following steps: inducing HPC generation and inducing T cell generation from HPCs, wherein the step of inducing HPC generation specifically includes: co-culturing the arterial endothelial cells with hemogenic endothelial cells.

Preferably, the arterial endothelial cells may be hPSC-derived autologous arterial endothelial cells.

Preferably, in the co-culture of arterial endothelial cells with hemogenic endothelial cells, the medium may be STEMdiff APEL 2 Medium (STEMCELL Technologies) supplemented with 50 ng/ml stem cell factor (SCF) (Peprotech), 50 ng/ml FMS-like tyrosine kinase 3 ligand (FLT3-L) (Peprotech), 5 ng/ml thrombopoietin (TPO) (Peprotech), 10 ng/ml interleukin 3 (IL-3) (Peprotech), 10 ng/ml vascular endothelial growth factor (VEGF) (Peprotech), 10 ng/ml basic fibroblast growth factor (bFGF) (Peprotech) and 10 μM SB-431542 (Selleck); the arterial endothelial cells and the hemogenic endothelial cells may have a co-culture ratio of 1:2 and the co-culture may be maintained at 37° C. under hypoxic conditions with 1%-5% 02 and the medium is changed every 2-3 days until day 7.

Preferably, the culture plates may be coated with 0.1 mg/ml Fibronectin (Corning) for 30 seconds before the co-culture.

Preferably, the arterial endothelial cells and the hemogenic endothelial cells may be obtained by the following steps: performing endothelial and hematopoietic differentiation using a stepwise monolayer system, where Day 0 to Day 2 of the differentiation is mesoderm formation and Day 2 to Day 5 of the differentiation is endothelial and hematopoietic specialization.

Specifically, the method for the generation of arterial endothelial cells and hemogenic endothelial cells may include the following steps:

mesoderm formation (Day 0 to Day 2):

wherein in this step, a medium is STEMdiff APEL 2 Medium (STEMCELL Technologies) supplemented with 3 μM CHIR99021 (abm), 2 ng/ml Activin A (Peprotech), 10 ng/ml bone morphogenetic protein 4 (BMP4) (Peprotech) and 10 μM Y-27632 (STEMCELL Technologies);

on Day 0, single hPSCs digested by TrypLE are plated at an optimized density of 1340 hPSC/cm²; and

endothelial and hematopoietic specialization:

wherein in this step, a basic medium is STEMdiff APEL 2 Medium (STEMCELL Technologies), where 10 ng/ml VEFG (Peprotech) is added on Day 2 and 10 ng/ml bFGF (abm) is added on Day 3; on the 5th day of differentiation, differentiated cells are digested by TrypLE (Gibco), and arterial endothelial cells (CD34+CD43−CD184+CD73+) and arterialized hemogenic endothelial cells (CD34+CD43−CD184+CD73-) are isolated by FACSAria III flow sorter (BD Biosciences).

On Day 0 of the mesoderm induction, the culture plates may be coated with 3.3 μg/ml Vitronectin (Peprotech) for 1 hour.

The whole differentiation process (Day 0 to Day 5) may be conducted at 37° C. under the 1%-5% hypoxic conditions.

The present disclosure has the following beneficial effects:

The present disclosure provides a method for mainly optimizing the first stage of T cell differentiation from hPSCs, where key autologous microenvironmental cells are found to enhance functional T cell generation by promoting the generation of HPCs with T-lineage bias. In the present disclosure, it is found that the arterial endothelial cells enhance the generation of HPCs with T-lineage bias, and the generated T cells have normal functions similar to peripheral blood (PB) T cells.

The autologous arterial endothelial cells have no potential immune rejection, which is more conducive to clinical transformation. In the present disclosure, the first stage of hPSC-T cell differentiation is optimized, and it is found that autologous arterial endothelial cells can increase the T-lineage differentiation potential of HPCs. The T cells primed by autologous arterial endothelial cells share similar functions to that of human PB T cells. The established hPSC-T differentiation system would provide a valuable resource for CAR-T therapy; hPSC-derived CD19-CAR-T have been verified to have potential tumor-killing effects both in vivo and in vitro. Generally, the method would further broaden the applications of CAR-T therapy to benefit patients, and would accelerate the clinical transformation to promote economic development.

### BRIEF DESCRIPTION OF THE DRAWINGS

FIG. 1 shows a strategy for generation and separation of autologous arterial endothelial cells.

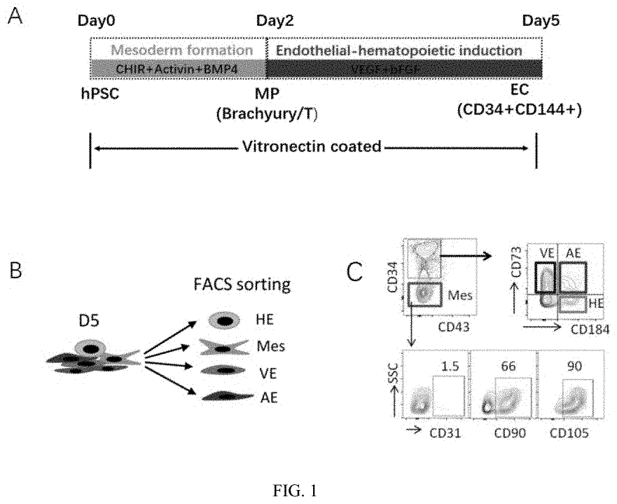

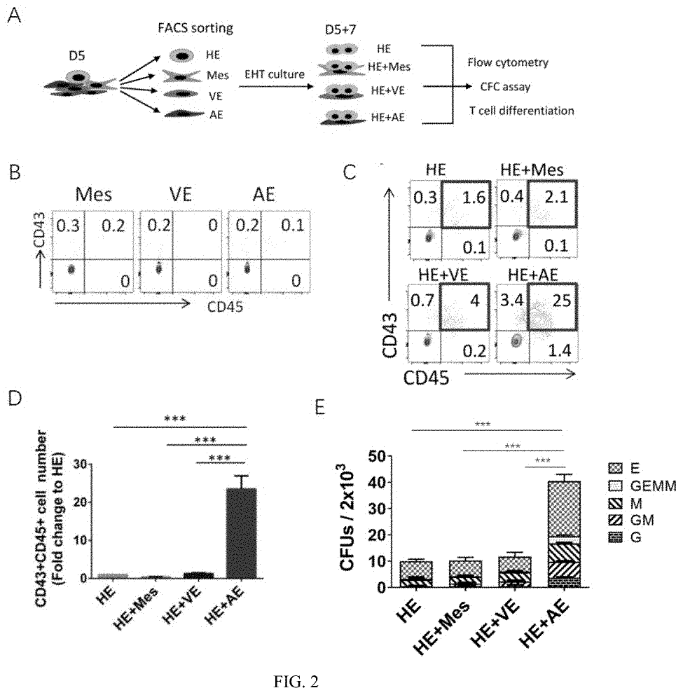

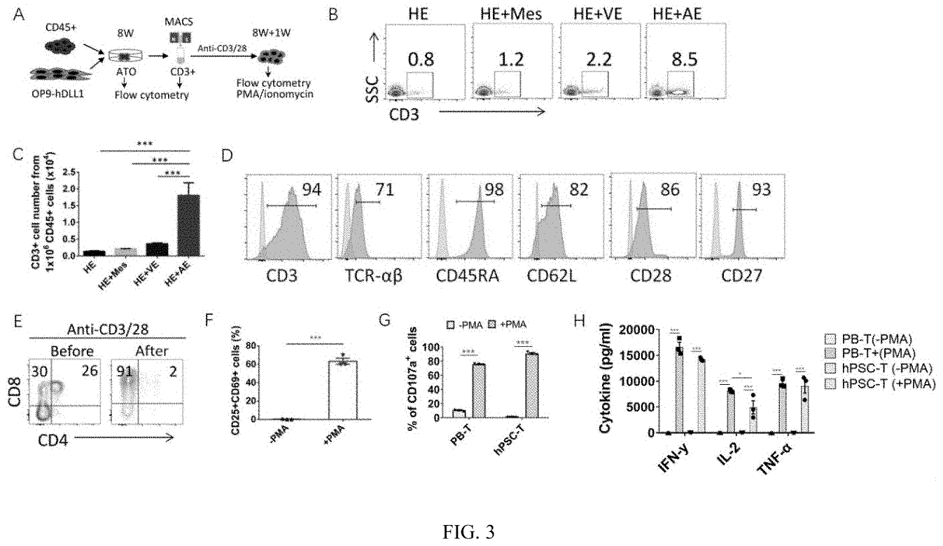

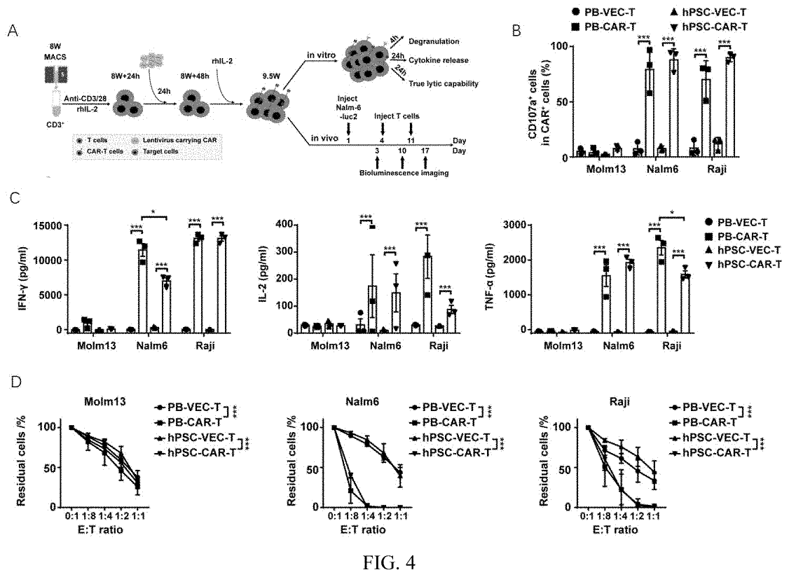

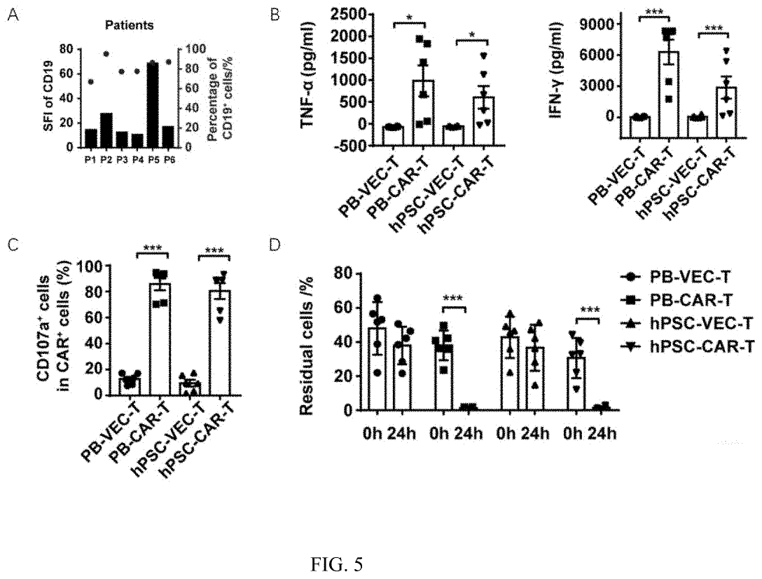

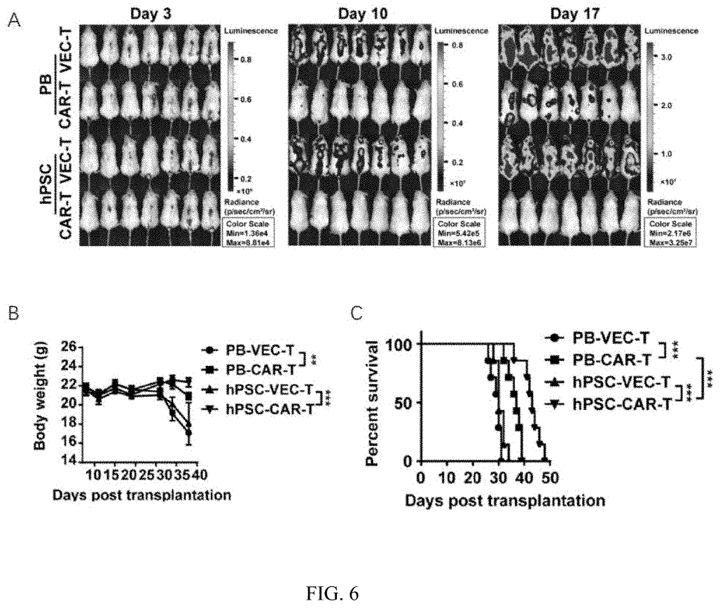

In FIG. 1, A is a schematic diagram of endothelial and hematopoietic differentiation, which is generally divided into two stages: Day 0 to Day 2 and Day 2 to Day 5. The Day 0 to Day 2 stage is a mesoderm induction stage, wherein Activin A, BMP4 and Wnt signaling pathway activator (CHIR99021) accelerate the production of Brachyury+ mesoderm progenitor cells (MP). The Day 2 to Day 5 stage is an endothelial and hematopoietic induction stage, wherein VEGF and bFGF directionally induce endothelial and hematopoietic differentiation from MP. B is a schematic diagram showing separation of autologous arterial endothelial cells. On the 5th day of differentiation, hemogenic endothelial cells (HE), mesenchymal cells (Mes), venous endothelial cells (VE) and arterial endothelial cells (AE) are isolated by flow cytometry, respectively. C is a specific sorting strategy for isolation of autologous arterial endothelial cells. HE are labelled with CD34+CD43−CD184+CD73−; Mes are labelled with CD34−CD43-CD31−CD90+CD105+; VE are labelled with CD34+CD43−CD184-CD73+; and AE are labelled with CD34+CD43−CD184+CD73+.

FIG. 2 shows an enhanced effect of autologous arterial endothelial cells on hematopoietic differentiation.

In FIG. 2, A is a schematic diagram showing a strategy for investigating an effect of arterial endothelial cells on hematopoiesis. Briefly, AE, VE, HE, or Mes are isolated from Day 5 differentiated cells and induced endothelial-to-hematopoietic transition (EHT) for 7 days (D5+7) with or without co-culture with AE, VE or Mes. B is representative flow cytometric analysis showing that during EHT, AE, VE, or Mes alone are unable to generate CD43+ and CD45+ hematopoietic cells, indicating that the absence of hematopoietic potential in these populations. C and D show that compared with the co-culture of HE with VE (HE+VE) or Mes (HE+Mes), HE alone is unable to initiate EHT efficiently, whereas the co-culture of HE with AE (HE+AE) for 7 days promotes EHT, significantly increasing the frequency (FIG. 2C) and total numbers (FIG. 2D) of CD43+ and CD45+HPCs. E shows colony forming potential of AE-primed HPCs. The total number of colonies is significantly increased in the HE+AE cultures, while there are no significant differences in the total number of colonies between the HE cultures and the HE+VE or HE+Mes cultures.

FIG. 3 shows an enhanced effect of arterial endothelial cells on the generation of T-lineage biased HPCs.

In FIG. 3, A is a schematic diagram of the protocol for T cell differentiation. Briefly, CD45+HPCs are co-cultured with OP9-hDLL1 cells in three-dimensional aggregates for 8 weeks. CD3+ T cells are sorted by magnetic-activated cell sorting (MACS) at week 8, and stimulated with anti-CD3/CD28 beads for another one week. B and C show that the isolated CD45+HPCs derived from AE co-cultures (HE+AE) give rise to increased frequency (FIG. 3B) and total numbers (FIG. 3C) of CD3+ T cells, whereas the T cell potential of CD45+HPCs from VE or Mes co-cultures, or without niche cells, is limited. D is representative flow cytometric analysis showing that the CD3+ T cells exhibit a naïve phenotype with the expression of αβ T cell receptor (TCR-αβ), CD45RA, CD62L, CD28, and CD27. E is representative flow cytometric analysis showing that before anti-CD3/CD28 treatment, CD8+CD4− cells and CD8+CD4+ cells are included among the CD3+ cells. After anti-CD3/CD28 treatment, CD8+CD4− cells account for more than 90% of the CD3+ cells. F is representative flow cytometric analysis showing that after stimulation with phorbol myristate acetate (PMA), CD25+CD69+ T cells significantly increased. G and H show that similar to PB T cells (PB-T), the hPSC-derived T cells (hPSC-T) generated from AE-primed HPCs are highly functional, releasing cytotoxic granules (CD107a expression) (FIG. 3G) with polyfunctional production of interferon-γ (IFN-γ), tumor necrosis factor-α (TNF-α), and interleukin-2 (IL-2) (FIG. 3H).

FIG. 4 shows in vitro tumor-killing potential of AE-primed hPSC-T following engineered expression of anti-CD19 CAR.

In FIG. 4, A is a schematic diagram showing the protocol for generating hPSC-CAR-T for cytotoxicity assays both in vitro and in vivo. The conventional CAR-T constructed from PB-isolated T cells (PB-CAR-T) are used as a positive control; hPSC-T and PB-T transfected with empty vectors (hPSC-VEC-T and PB-VEC-T) are used as negative controls. CD19+ cell lines (Nalm-6 and Raji) and a CD19− cell line (Molm13) are used as target cells. B shows that hPSC-CAR-T and PB-CAR-T show significant cytotoxic granule release (CD107a expression) when co-cultured with Nalm-6 and Raji cells, while little CD107a up-regulation is observed following co-culture with Molm13 cells. C shows that secretion of IL-2, TNF-α, and IFN-γ is significantly increased in both hPSC-CAR-T and PB-CAR-T when co-cultured with CD19+ target cells. D shows that hPSC-CAR-T kill CD19+ leukemia cells as effectively as PB-CAR-T in vitro.

FIG. 5 shows in vitro primary tumor-killing potential of AE-primed hPSC-CAR-T.

In FIG. 5, A shows the expression of CD19 in bone marrow cells of 6 patients with B-ALL. B, C and D show that hPSC-CAR-T and PB-CAR-T share similar cytotoxic characteristics against CD19+ target cells, including cytokine release (TNF-α and IFN-γ) (FIG. 5B), degranulation ability (CD107a expression) (FIG. 5C), and true lytic capability (FIG. 5D).

FIG. 6 shows in vivo tumor-killing potential of AE-primed hPSC-CAR-T.

In FIG. 6, A is bioluminescent imaging showing that hPSC-CAR-T and PB-CAR-T can significantly inhibit tumor growth in vivo. B shows that hPSC-CAR-T can significantly delay weight loss of the mouse in vivo. C is a survival curve of the mice showing that hPSC-CAR-T can significantly prolong the survival of the treated mice.

### DETAILED DESCRIPTION OF THE EMBODIMENTS

The present disclosure is further described below with reference to specific examples, which are not intended to unduly constrain the protection scope of the present disclosure.

1. Induction and Isolation of Autologous Arterial Endothelial Cells and Hemogenic Endothelial Cells

A monolayer-based, stepwise culture system was applied to direct H1-ESC differentiation toward the endothelial and hematopoietic lineages. The E8 medium (Life Technologies) was used for H1 culture; 10 μM Y-27632 (STEMCELL Technologies) was added during recovery or passage and was removed within 24 hours. H1 cells were cultured on Matrigel (BD) diluted at 1:90. A recovery method of H1 cells was as follows: H1 cells were carefully and quickly removed from liquid nitrogen, and placed in a 37° C. water bath to quickly melt; cells in a cryopreservation tube were added dropwise to the E8 medium, and centrifuged at 1,000 rpm for 5 minutes; a supernatant was discarded, cell pellet after centrifugation was blew off by using 1 ml E8 medium+Y-27632 (E8+Y) medium, and the cells were inoculated into Matrigel-coated plates. Medium was renewed every day, and the cells were passaged or ready for differentiation when the confluence of cells reached 70%-80%. Digestion solution for H1 cell passage was 0.5 mM ethylene diamine tetraacetic acid (EDTA) (Gibco). Digestion was conducted for 3-5 minutes at room temperature. The EDTA was aspirated and the cells were blew off using the E8+Y, and finally the cells were added to a new culture plate coated with Matrigel. The above method was also applicable to other hPSC cultures. Here and below, H1 cells were used as an example.

For differentiation, single-cell suspensions of H1 were obtained by treating the cultures at 70%-80% confluency with TrypLE (Gibco). Day 0 to Day 2 was the stage of mesoderm formation. Activin A, BMP4 and Wnt signaling pathway activator (CHIR99021) could accelerate the production of Brachyury+ mesodermal progenitor cells. The medium used for mesoderm formation on Day 0 to Day 2 was STEMdiff APEL 2 Medium (Stem Cell Technologies) supplemented with 3 μM CHIR99021 (abm), 2 ng/ml Activin A (Peprotech), 10 ng/ml BMP4 (Peprotech) and 10 μM Y-27632 (STEMCELL Technologies). On Day 0, the culture plates were coated with 3.3 μg/ml Vitronectin (Peprotech) for 1 hour, and then Vitronectin was replaced with the differentiation medium of Day 0 to Day 2. Single cells were then plated at an optimized density of 1340 hPSC/cm². Day 2 to Day 5 was the stage of endothelial and hematopoietic specialization. VEGF and bFGF induced mesodermal progenitor cells to gradually differentiate to endothelial and hematopoietic cells (FIG. 1-A). A basic medium used at this step was STEMdiff APEL 2 Medium (STEMCELL Technologies), where 10 ng/ml VEGF (Peprotech) was added on Day 2, and 10 ng/ml bFGF (abm) was added on Day 3. The whole differentiation process (Day 0 to Day 5) was conducted at 37° C. in a 1%-5% hypoxic environment.

On the 5th day of differentiation, the differentiated cells were digested with TrypLE (Gibco) and different cell components were isolated by FACSAria III flow sorter (BD Biosciences), including AE (CD34+CD43−CD184+CD73+), VE (CD34+CD43−CD184−CD73+), HE (CD34+CD43−CD184+CD73−) and Mes (CD34−CD43−CD31−CD90+CD105+) (FIG. 1-B to FIG. 1-C).

Collectively, in this part, a monolayer-based system was developed for autologous arterial endothelial and hematopoietic differentiation. AE (CD34+CD43-CD184+CD73+) and HE (CD34+CD43-CD184+CD73-) were isolated by using flow sorting.

2. Arterial Endothelial Cell Enhanced the Generation of HPCs with T-Lineage Bias

To further determine the roles of arterial cells and other cellular niches in hematopoiesis, AE, VE, HE, or Mes were isolated from Day 5 differentiated H1 hESCs and induced EHT for 7 days (D5+7) with or without co-culture (FIG. 2-A). During the co-culture, STEMdiff APEL 2 Medium (STEMCELL Technologies) was applied, supplemented with 50 ng/ml SCF (Peprotech), 50 ng/ml FLT3-L (Peprotech), 5 ng/ml TPO (Peprotech), 10 ng/ml IL-3 (Peprotech), 10 ng/ml VEGF (Peprotech), 10 ng/ml bFGF (Peprotech) and 10 μM SB-431542 (Selleck). The culture plates were coated with 0.1 mg/ml Fibronectin (Corning) for 30 seconds before the co-culture. For co-culturing, HE was co-cultured with AE, VE or Mes at 2:1. Cultures were maintained at 37° C. under hypoxic conditions with 1% O₂ and the medium was changed every 2-3 days until Day 7. On D5+7, the cells were digested with TrypLE (Gibco) for further flow cytometric analysis, CFC assay and T cell differentiation. The results showed that during EHT, AE, VE, or Mes alone were unable to generate CD43+ and CD45+ hematopoietic cells, indicating that the absence of hematopoietic potential in these populations (FIG. 2-B). Compared with the co-culture of HE cells with VE (HE+VE) or Mes (HE+Mes), HE cells alone were unable to initiate EHT efficiently, whereas the co-culture of HE cells with AE (HE+AE) for 7 days promoted EHT, significantly increasing the frequency and total numbers of CD43+ and CD45+HPCs (FIG. 2-C to FIG. 2-D), and significantly increased the number of hematopoietic colonies (FIG. 2-E).

To further investigate the effect of arterial endothelial cells on T cell differentiation, T cell differentiation from HPCs was performed. CD45+HPCs from different sources, including HE-derived, AE+HE-derived, VE+HE-derived and Mes+HE-derived, were sorted by MACS. For T cell differentiation, 5×10⁵ CD45+ cells were centrifuged with 1×10⁶ OP9-hDLL1 cells at a ratio of 1:2 to form small 3D aggregates, which were then plated onto a 0.4-mm Millicell transwell insert (EMD Millipore) placed in a 6-well plate containing 1 mL T cell differentiation medium consisting of RPMI 1640 (Gibico), 4% B27 supplement (ThermoFisher Scientific), 30 mM L-ascorbic acid 2-phosphate sesquimagnesium salt hydrate (Sigma-Aldrich), 1% penicillin/streptomycin (ThermoFisher Scientific), 20 ng/ml SCF (Peprotech), 5 ng/ml FLT3L (Peprotech) and 5 ng/ml IL-7 (Peprotech). During T cell differentiation, the medium was changed every 3-4 days. After 8 weeks of differentiation, the cells were digested with TrypLE (Gibco) for flow cytometric analysis, and CD3+ T cells were enriched by MACS (FIG. 3-A). T cell-related markers were detected at week 8. It was found that AE-primed CD45+HPCs had significantly improved T cell differentiation potential to generate a higher proportion and number of CD3+ TCRαβ+ T cells (FIG. 3-B to FIG. 3-D). Further study revealed that the CD3+ TCRαβ+ T cells exhibited a naïve phenotype with the expression of CD45RA, CD62L, CD28 and CD27 (FIG. 3-D). Before anti-CD3/CD28 treatment, CD8+CD4− cells and CD8+CD4+ cells were included among the CD3+ TCRαβ+ T cells. After anti-CD3/CD28 treatment, CD8+CD4− cells accounted for more than 90% of the CD3+ TCRαβ+ T cells (FIG. 3-E). To examine whether the T cells derived from AE-primed HPCs in vitro are functional, the CD3+CD4-CD8+ cells are simulated in the presence of the T cell activator PMA-ionomycin, and quantitated surface marker expression and cytokine production. After the stimulation, CD25+CD69+ T cells significantly increased (FIG. 3-F). Similar to PB-T, the hPSC-T generated from AE-primed HPCs were highly functional, releasing cytotoxic granules (CD107a expression) (FIG. 3-G) with polyfunctional production of IFN-γ, TNF-α, and IL-2 (FIG. 3-H).

3. Arterial Endothelial Co-Culture Promotes HPCs to Obtain Functional T Cell Potential

To further evaluate the tumor-killing potential of T cells derived from AE-primed HPCs, the T cells were engineered to express anti-CD19 CAR (hPSC-CAR-T) for cytotoxicity assays both in vitro and in vivo. The conventional CAR-T constructed from PB isolated T cells (PB-CAR-T) were used as a positive control; hPSC-T and PB-T transfected with empty vectors (hPSC-VEC-T and PB-VEC-T) were used as negative controls (FIG. 4-A). T cell culture medium was X-VIVO 15 medium (Lonza) supplemented with 5% fetal bovine serum (FBS) (Gibco) and 50-100 U/ml rhIL-2 (Peprotech). Human B-ALL cell line Nalm-6 and lymphoma cell line Raji were used as CD19+ target cells, and human acute myeloid leukemia cell line Molm13 was used as CD19− target cells. Tumor cell culture medium was RPMI-1640 medium supplemented with 10% FBS (Gibco). The abilities of degranulation, cytokine releasing and direct cytotoxicity of hPSC-CAR-T were detected through co-culture of CAR-T cells with tumor cells. The hPSC-CAR-T, PB-CAR-T, hPSC-VEC-T or PB-VEC-T were co-cultured with Nalm-6, Raji or Molm13 tumor cells at a ratio of 1:1. After 5 hours, the degranulation ability (CD107a expression) of T cells was detected by flow cytometry. After 24 hours, the secretion of IL-2, IFN-γ and TNF-α was detected by enzyme-linked immunosorbent assay (ELISA) kit (R&D, USA), and the residual CD19+ tumor cells were detected by flow cytometry. The results showed that the hPSC-CAR-T cells were significantly activated after stimulation by CD19+ leukemia cells in vitro, including degranulation (CD107a+) (FIG. 4-B) and the secretion of IFN-γ, IL-2 and TNF-α (FIG. 4-C). Moreover, the hPSC-CAR-T also showed a strong tumor-killing potential in vitro (FIG. 4-D).

To further clarify the tumor-killing potential of T cells, the cytotoxicity of hPSC-CAR-T and PB-CAR-T in primary B-ALL cells was compared. Bone marrow mononuclear cells (BMMNCs) collected from six B-ALL patients were used as target cells. (FIG. 5-A). The hPSC-CAR-T, PB-CAR-T, hPSC-VEC-T or PB-VEC-T were co-cultured with primary B-ALL cells at a ratio of 1:1. After 5 hours, the degranulation ability (CD107a expression) of T cells was detected by flow cytometry. After 24 hours, the secretion of IFN-γ and TNF-α was detected by ELISA (R&D, USA), and the residual CD19+ tumor cells were detected by flow cytometry. It was showed that hPSC-CAR-T and PB-CAR-T cells shared similar cytotoxic characteristics against CD19+ target cells, including degranulation ability, cytokine release (FIG. 5-B to FIG. 7-C). Moreover, the hPSC-CAR-T cells showed high tumor-killing potential similar to the PB-CAR-T in vitro (FIG. 5-D).

Finally, the cytotoxic efficacy of hPSC-CAR-T toward CD19+B-ALL in vivo was evaluated by establishing a B-ALL mouse model. A total of 3×10⁵ luciferase-expressing Nalm-6 cells (Nalm-6-luc2) were intravenously injected into NOD/SCID mice. At Days 4 and 11, 5×10⁶ PB-VEC-T, hPSC-VEC-T, PB-CAR-T or hPSC-CAR-T cells were administered intravenously. Bioluminescent imaging showed that the injection of hPSC-CAR-T inhibited tumor progression (FIG. 6-A), delayed weight loss (FIG. 6-B) and prolonged the survival of the treated mice (FIG. 6-C). Using the same protocol, it was showed that the therapeutic performance of hPSC-CAR-T and PB-CAR-T were comparable.

Collectively, it demonstrates that hPSC-derived arterial endothelial cells enhance the generation of HPCs with more T cell potential. The T cells generated from arterial endothelium-primed HPCs have normal functions to inhibit tumor growth both in vitro and in vivo.

What is claimed is:

1. Use of arterial endothelial cells in enhancing functional T cell generation.

2. The use according to claim 1, wherein the arterial endothelial cells enhance functional T cell generation by promoting generation of hematopoietic progenitor cells (HPCs) with T-lineage bias.

3. The use according to claim 1, wherein the arterial endothelial cells are autologous arterial endothelial cells.

4. The use according to claim 1, wherein the arterial endothelial cells are human pluripotent stem cell (hPSC)-derived autologous arterial endothelial cells.

5. A method for arterial endothelial-enhanced functional T cell generation, comprising the following steps: inducing HPC generation and inducing T cell differentiation from HPCs, wherein the step of inducing HPC generation comprises: co-culturing the arterial endothelial cells with hemogenic endothelial cells.

6. The method according to claim 5, wherein the arterial endothelial cells are hPSC-derived autologous arterial endothelial cells.

7. The method according to claim 5, wherein co-culturing the arterial endothelial cells with hemogenic endothelial cells is conducted in a medium that comprises STEMdiff APEL 2 Medium supplemented with 50 ng/ml stem cell factor (SCF), 50 ng/ml FMS-like tyrosine kinase 3 ligand (FLT3-L), 5 ng/ml thrombopoietin (TPO), 10 ng/ml interleukin 3 (IL-3), 10 ng/ml vascular endothelial growth factor (VEGF), 10 ng/ml basic fibroblast growth factor (bFGF) and 10 μM SB-431542; for co-culturing, the arterial endothelial cells are co-cultured with hemogenic endothelial cells at a ratio of 1:2; and the co-cultures are maintained at 37° C. under hypoxic conditions with 1%-5% 02 and the medium is changed every 2-3 days until day 7.

8. The method according to claim 7, wherein culture plates are coated with 0.1 mg/ml Fibronectin for 30 seconds before the co-culturing.

9. The method according to claim 7, wherein the arterial endothelial cells and the hemogenic endothelial cells are obtained by a differentiation process including the following steps:
mesoderm formation (Day 0 to Day 2),
in which single hPSCs digested by TrypLE are plated at an optimized density of 1340 hPSC/cm² in STEMdiff APEL 2 Medium supplemented with 3 μM CHIR99021, 2 ng/ml Activin A, 10 ng/ml bone morphogenetic protein 4 (BMP4) and 10 μM Y-27632; and
endothelial and hematopoietic specialization,
in which a basic medium is STEMdiff APEL 2 Medium, wherein 10 ng/ml VEFG is added on Day 2 and 10 ng/ml bFGF is added on Day 3; on a 5th day of differentiation, differentiated cells are digested by TrypLE, and arterial endothelial cells (CD34+CD43−CD184+CD73+) and arterialized hemogenic endothelial cells (CD34+CD43−CD184+CD73-) are isolated by FACSAria III flow sorter;
wherein on Day 0 of the mesoderm induction, culture plates are coated with 3.3 μg/ml Vitronectin for 1 hour.

10. The method according to claim 9, wherein the differentiation process from Day 0 to Day 5 is conducted at 37° C. under the 1%-5% hypoxic conditions.
